(19) United States
(12) Patent Application Publication
(10) Pub. No.: US 2019/0193063 A1
(43) Pub. Date: Jun. 27, 2019

(54) CATALYSTS SUPPORTED ON NATURAL POLYMERS FOR THE PRODUCTION OF CARBONATES FROM CO2

(71) Applicant: INSTITUTO MEXICANO DEL PETRÓLEO, Mexico City (MX)

(72) Inventors: Heriberto DÍAZ VELÁZQUEZ, Mexico City (MX); Rafael MARTÍNEZ PALOU, Mexico City (MX); Javier GUZMÁN PANTOJA, Mexico City (MX); Roberto GARCÍA DE LEÓN, Mexico City (MX)

(21) Appl. No.: 15/631,089

(22) Filed: Jun. 23, 2017

(30) Foreign Application Priority Data
Jun. 30, 2016 (MX) MX/A/2016/008702

(51) Int. Cl. B01J 31/26; B01J 31/02; B01J 27/08; B01J 37/04; B01J 37/02; B01J 31/06; C07D 317/36

(52) U.S. Cl. CPC B01J 31/26; B01J 31/0237; B01J 31/0241; B01J 31/0244; B01J 27/08; B01J 2231/48; B01J 37/0236; B01J 31/061; C07D 317/36; B01J 2531/002; B01J 37/04

(57) ABSTRACT

The present invention describes a process to prepare catalyst systems based on metal salts, supported on natural polymers and co-catalyzed by organic bases, for the catalytic transformation of carbon dioxide to organic carbonates through cycloaddition reactions to epoxides. The advantages of the presented system can be summarized on the use of raw materials of low cost for the preparation of the catalyst system, minimal environmental risk due to the low toxicity of the materials used, in some cases biodegradable such as the natural polymers, as well as high catalytic efficiency, reaching selectivities up to 100% and in some cases quantitative yields.

### TECHNICAL FIELD OF THE INVENTION

The present invention describes the synthesis of cyclic carbonates through the reaction of carbon dioxide and epoxides, using natural polymers as catalysts supports, which are formed by inorganic salts and organic bases (amines).

### BACKGROUND OF THE INVENTION

Carbon dioxide (CO₂) is the main gas component responsible for the greenhouse effect, for that reason it has gained great attention regarding his sequestration and chemical transformation from flue gas. Among the chemical transformation processes for CO₂ we can cite the production of several materials of chemical value such as: dimethyl carbonate, cyclic carbonates, polycarbonates, substituted ureas, aretanes, etc. Cyclic carbonates are also widely used in the production of engineering plastics such as polycarbonates and polyurethanes, electrolytes in lithium batteries, intermediates in fine chemistry and pharmaceutics. Generally, they are also used as polar aprotic solvents in chemical synthesis.

The synthesis methods used for cyclic carbonates are: the use of olefins as raw materials in a process called Oxidative Carboxylation or 2. Cycloaddition of CO₂ to epoxides.

The cycloaddition of CO₂ to epoxides is performed with high efficiency and its heterogeneous nature is relatively easy for the catalyst to be reused in several reaction cycles. Among the most common catalysts for this reaction, the alkaline metal salts are convenient for their low price, high availability and easily reused. However, their activity is low in some cases. To solve this problem, co-catalysts such as crown ethers, phenol or porphyrins are used.

There is an ample literature available about synthesis of organic carbonates because of the appealing advantages of the method. Research is advancing in this direction because the use of CO₂ as abundant and renewable carbon source avoids the use of a toxic and environmentally harming product such as phosgene. Organic carbonates, especially cyclic ones, are commercially important and their applicability makes them useful in diverse areas of pharmaceutical and chemical industry, such as the plastic manufacture, electrolyte solvents for lithium batteries, organic solvents, green reagents and fuel additives. Commonly, cyclic carbonates area synthesized using epoxides, diols and olefins as raw material, using expensive metal catalysts and severe reaction conditions.

In an article “Conversion of Carbon Dioxide and Olefins into Cyclic Carbonates in Water”, Nicolas Eghbali develops a process to transform alkenes and CO₂ into cyclic carbonates using N-Bromosuccinimide and 1,8-diazabiciclo[5.4.1]-7-undecene in water or by using a small amount of Bromide and aqueous hydrogen peroxide:

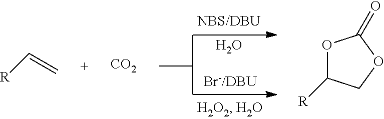

In the article “Efficient Synthesis of Cyclic Carbonates from CO₂ and Epoxides over Cellulose/KI” (Chem. Commun, 2011, 47, 2131-2133), a new active, selective and stable catalyst system based on Cellulose/KI is reported. This recyclable catalyst was highly for the cycloaddition reaction of CO₂ to epoxides as is shown:

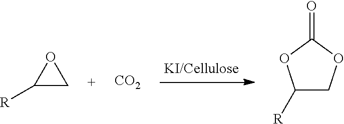

In the article “Super base/cellulose: an environmentally benign catalyst for Chemical Fixation of Carbon Dioxide into Cyclic Carbonates” (Green Chem. 2014, 16, 3071-3078), the group of Prof. Zhang shows how the use of DBU in conjunction with cellulose represents an excellent catalyst system for the transformation of epoxides to cyclic carbonates by CO₂ addition reaction as is shown:

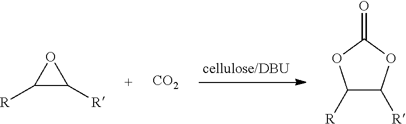

In the review entitled “Transformation of CO₂” (Chem. Rev. 2007, 107, 2365-2387), Sakakura makes a summary of all the existing methods for the synthesis of cyclic carbonates from diverse materials such as those shown:

### From Olefins

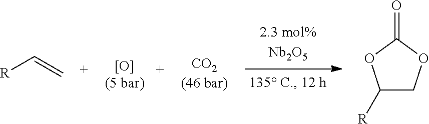

### From Diols

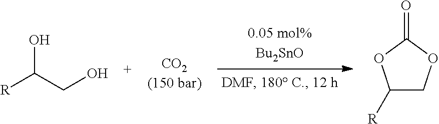

### From Cyclic Ketals

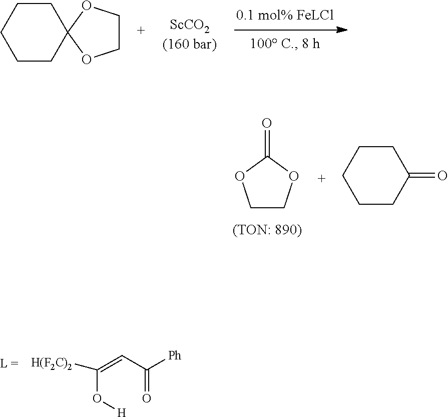

From Propargyl Alcohols

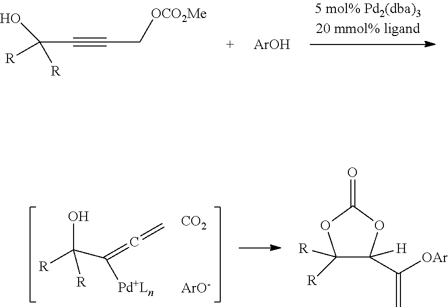

The U.S. Pat. No. 6,870,379 B2 reveals a catalyst system composed by a substituted polyoxometalate which, along with a Lewis base, were shown to be efficient for the cycloaddition of CO₂ to epoxides to form cyclic carbonates.

The U.S. Pat. No. 8,937,197 describes a process to produce organic carbonates via the reaction of carbon monoxide adsorbed on gold particles and alcohols.

The Chinese patent CN102188999B reports the preparation of an ionic liquid catalyst functionalized with the mesoporous material Al-MCM-41 for the production of propylene carbonate.

The Chinese patent EP2468402 reveals a supported catalyst, which use a nanostructured material as support, containing a phosphonium salt, whose molecular weight is about 1,000-2,000 for the cycloaddition of carbon dioxide to epoxides.

The US patent US2011019616 explores a method to produce organic carbonates such as dimethyl carbonates using a heterogeneous catalyst containing both an acid and a basic moiety.

The Chinese patent CN101966460B explains the invention of a catalyst system made from Cu and Fe salts and a co-adjuvant containing whether Ag, In, Y, V, Pd, Ce, Mo, Co or these metals oxides to produce organic carbonates.

The patent EP2283003 describes a process for the production of cyclic carbonates through the reaction of epoxides with CO2, in presence of a catalyst containing phosphonium salts, that are continuously introduces in the reaction zone and separated through the product stream.

The patent EP2226328 describes a process to prepare cyclic carbonates by the reaction of epoxies and CO₂ using tin oxides containing alkyl groups in the complex structure.

The patent CN101376632 is related to a method to prepare propylene carbonate starting from CO₂ and 1, 2-propilene glycol. This method uses DBU as catalyst and Acetonitrile as solvent. Such reaction is performed during 12-15 h, with a temperature of 215 to 235° C. and pressure of 12-14 MPa. The conversion was only 67% and a yield of 83%.

The patent WO200884086 A1 is related to a method for the production of o-vinyl carbamates or vinyl-carbonates via the reaction of a secondary amine or alcohol with an alkyne in the presence of a catalyst selected from carbonyl complexes of Re, Mn, W, Mo, Cr and Fe at 25° C. temperature.

The patent CN101164696 relates the invention of catalyst system for the preparation of propylene carbonate applying a catalyst “QCS-N” constituted by a chitosan molecules functionalized with an ammonium salt, using the ammonium groups as the anchor for the active group for the transformation.

The US patent US 2006/0094893 A1 relates a process for the preparation of organic carbonates, by the reaction of an epoxides with CO₂ in presence of a catalyst based on titanosilicates and a base as co-catalyst under smooth reaction conditions (40° C. and 2 bar).

The U.S. Pat. No. 7,365,214 B2 accounts for an improved process for the preparation of cyclic carbonates including the reaction of olefins or their epoxides with CO₂ in the presence of a catalyst based on zeolites and a Lewis base as co-catalyst, under smooth reaction conditions (2bar) and a temperature range of 40-120° C.

The European patent WO 2011159219 A1 describes a process for the production of organic cyclic carbonates from a diol, triol or polyol and a CO₂ source such as dialkyl-carbonate. Such process is carried out using a NHC-metal complex as catalyst, under a solvent-free process. This process comprise a trans-esterification and a disproportion step.

As is possible to visualize, several methods have been developed for the production of cyclic carbonates; however, serious disadvantages exist respective to those using transition metals due to their known toxicity for humans and the environment. Furthermore, there exist procedures that do not require severe conditions of pressure and temperature , such as those that use principally aluminum and tin complexes, however, due to their high reactivity, it would not be possible to design a scaling-up procedure using these compounds because inert conditions would be necessary to realize such process. In the other hand, the majority of the described processes use solvents that represent a risk for living matter. For this reason it is imperative the development of eco-friendly catalysts systems and capable to produce organic carbonates with high efficiency, under smooth conditions and using solvent as low as possible.

### BRIEF DESCRIPTION OF THE FIGURES OF THE INVENTION

The accompanying drawings are part of the present disclosure and are incorporated into the specification. The drawings illustrate examples of embodiments of the disclosure and, in conjunction with the description and claims, serve to explain various principles, features, or aspects of the disclosure. Certain embodiments of the disclosure are described more fully below with reference to the accompanying drawings. However, various aspects of the disclosure may be implemented in many different forms and should not be construed as being limited to the implementations set forth herein. Like numbers refer to like, but not necessarily the same or identical, elements throughout.

FIG. 1 shows gas chromatograph data for a reaction that converts CO₂ to cyclic carbonates, in accordance with an embodiment of the present disclosure.

FIG. 2 shows gas chromatograph data for a reaction that converts CO₂ to cyclic carbonates, in accordance with an embodiment of the present disclosure.

FIG. 3 shows gas chromatograph data for a reaction that converts CO₂ to cyclic carbonates, in accordance with an embodiment of the present disclosure.

FIG. 4 shows gas chromatograph data for a reaction that converts CO₂ to cyclic carbonates, in accordance with an embodiment of the present disclosure.

FIG. 5 shows gas chromatograph data for a reaction that converts CO₂ to cyclic carbonates, in accordance with an embodiment of the present disclosure.

FIG. 6 shows gas chromatograph data for a reaction that converts CO₂ to cyclic carbonates, in accordance with an embodiment of the present disclosure.

FIG. 7 shows gas chromatograph data for a reaction that converts CO₂ to cyclic carbonates, in accordance with an embodiment of the present disclosure.

FIG. 8 shows gas chromatograph data for a reaction that converts CO₂ to cyclic carbonates, in accordance with an embodiment of the present disclosure.

FIG. 9 shows gas chromatograph data for a reaction that converts CO₂ to cyclic carbonates, in accordance with an embodiment of the present disclosure.

FIG. 10 shows gas chromatograph data for a reaction that converts CO₂ to cyclic carbonates, in accordance with an embodiment of the present disclosure.

FIG. 11 shows gas chromatograph data for a reaction that converts CO₂ to cyclic carbonates, in accordance with an embodiment of the present disclosure.

FIG. 12 shows gas chromatograph data for a reaction that converts CO₂ to cyclic carbonates, in accordance with an embodiment of the present disclosure.

FIG. 13 shows gas chromatograph data for a reaction that converts CO₂ to cyclic carbonates, in accordance with an embodiment of the present disclosure.

FIG. 14 shows gas chromatograph data for a reaction that converts CO₂ to cyclic carbonates, in accordance with an embodiment of the present disclosure.

FIG. 15 shows gas chromatograph data for a reaction that converts CO₂ to cyclic carbonates, in accordance with an embodiment of the present disclosure.

FIG. 16 shows the influence of the different natural polymer support that can be sued in this reaction, in accordance with an embodiment of the present disclosure.

FIG. 17 shows the effect of a metal halide over the yield of the reaction of CO₂ and propylene oxide, using chitosan as support, in accordance with an embodiment of the present disclosure.

FIG. 18 shows the influence of the mass composition on the catalyst KI/DBU, in the yield of this reaction, in accordance with an embodiment of the present disclosure.

FIG. 19 shows the yield evolution from the reaction after each catalytic cycle, in accordance with an embodiment of the present disclosure.

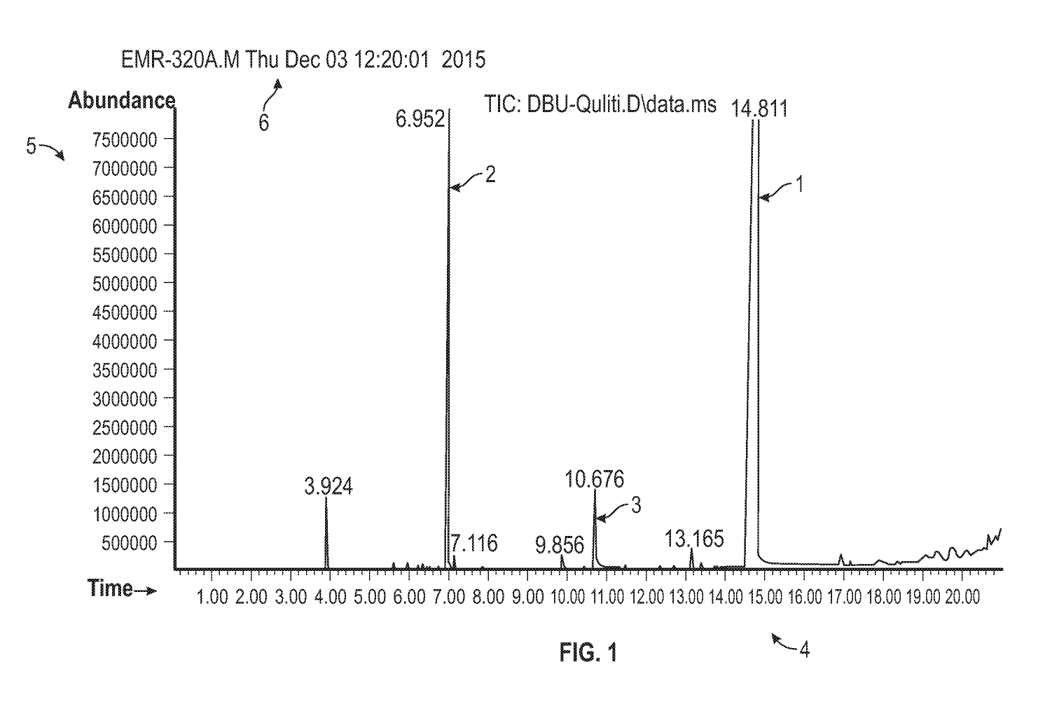

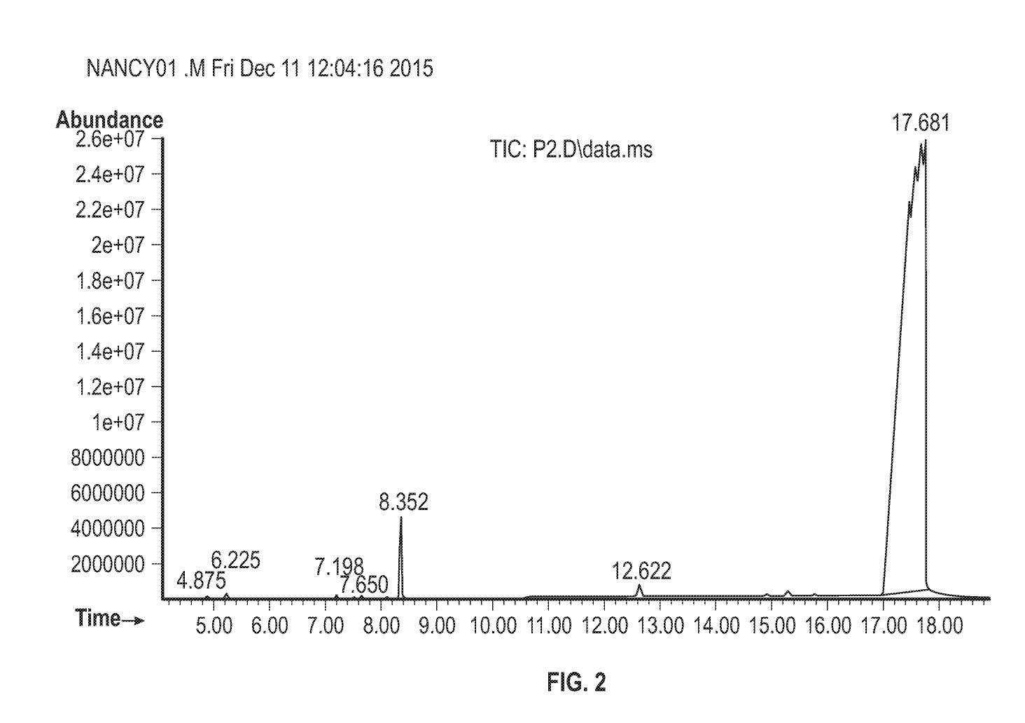

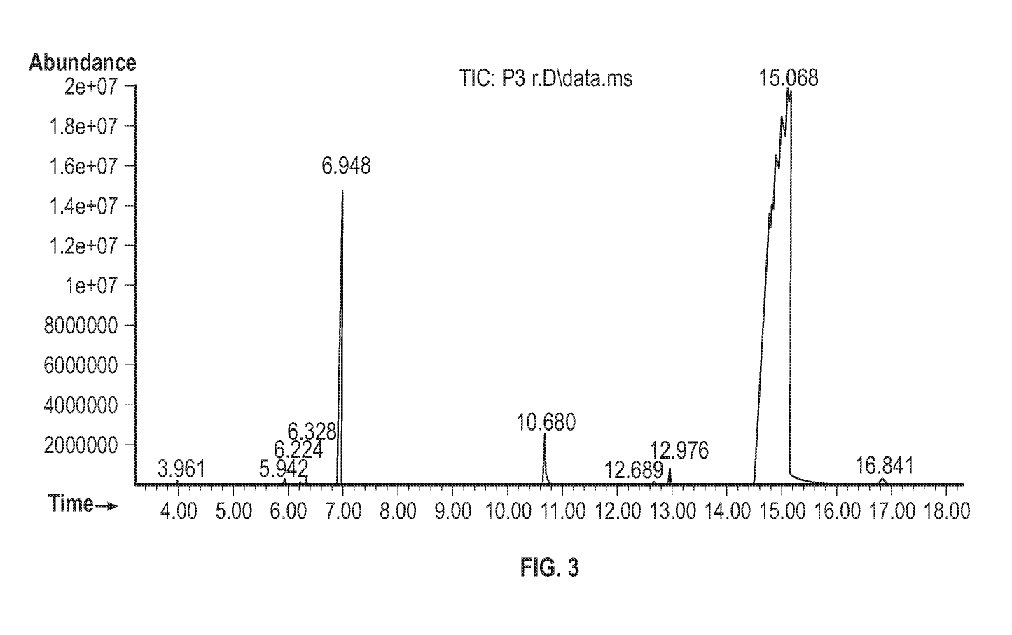

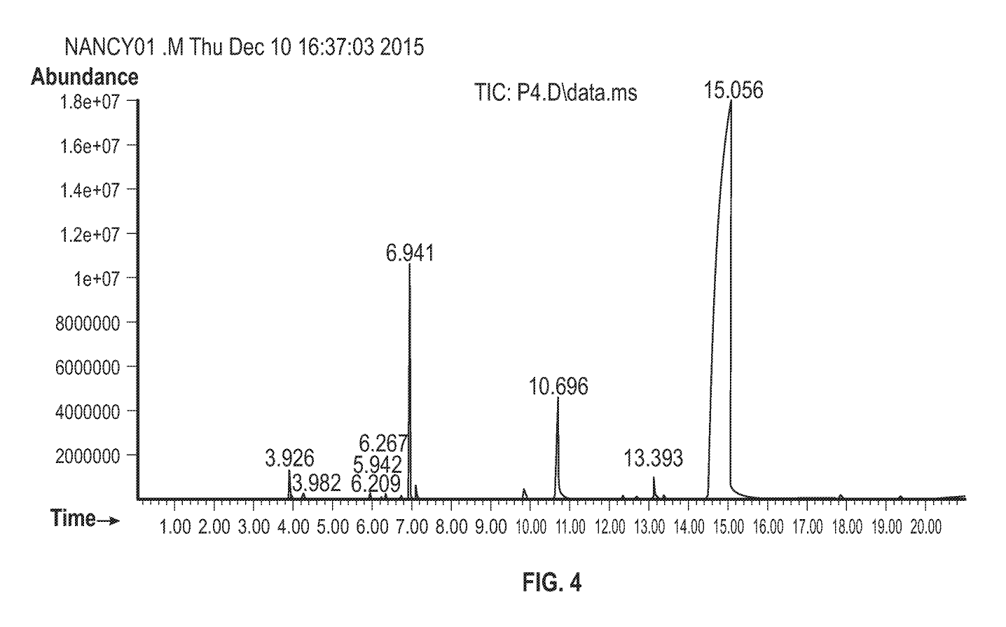

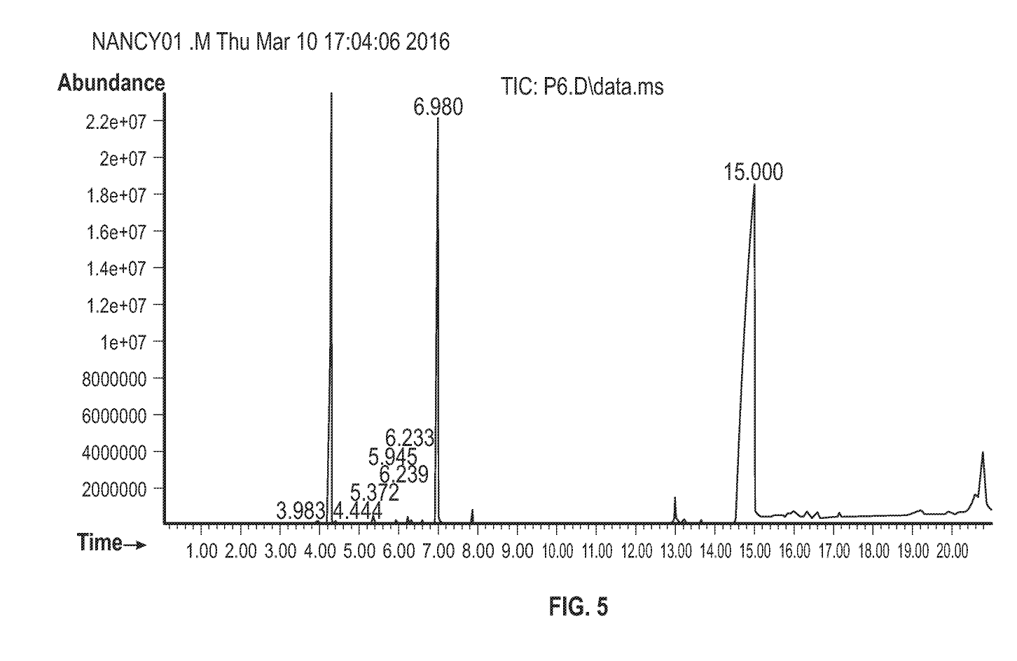

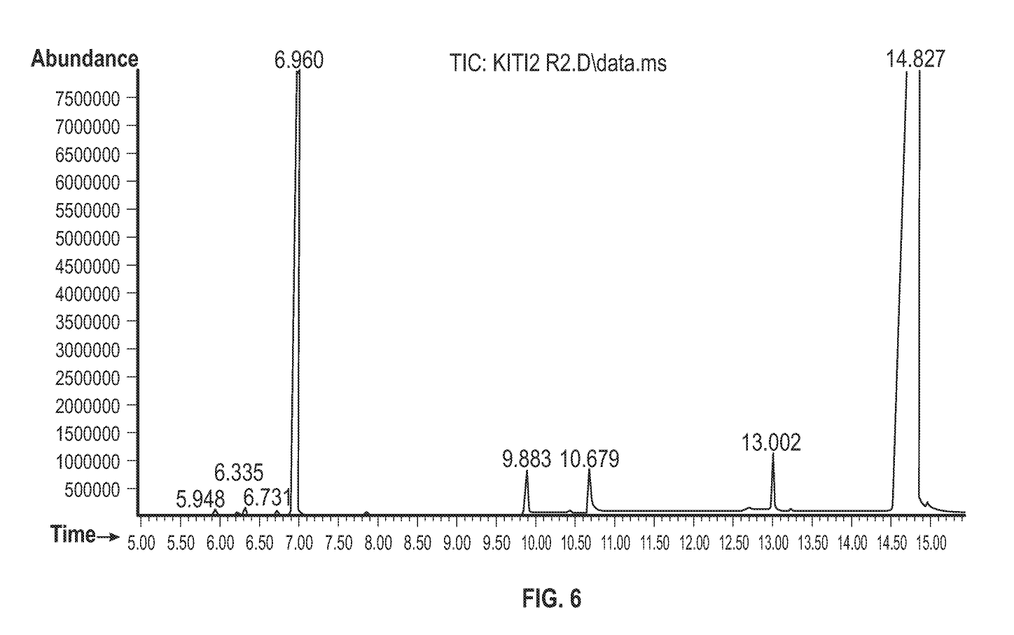

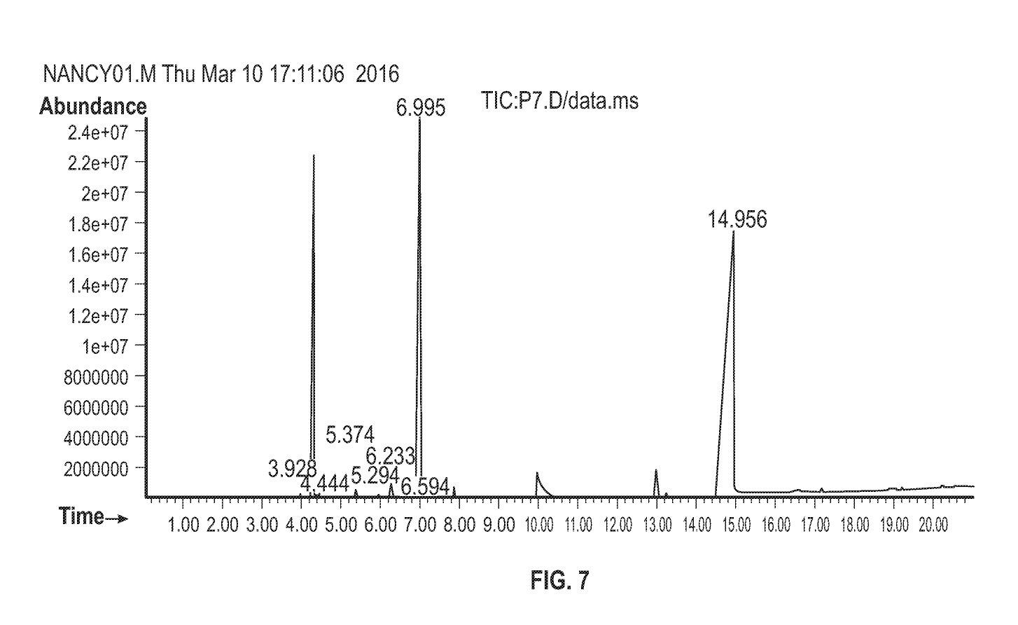

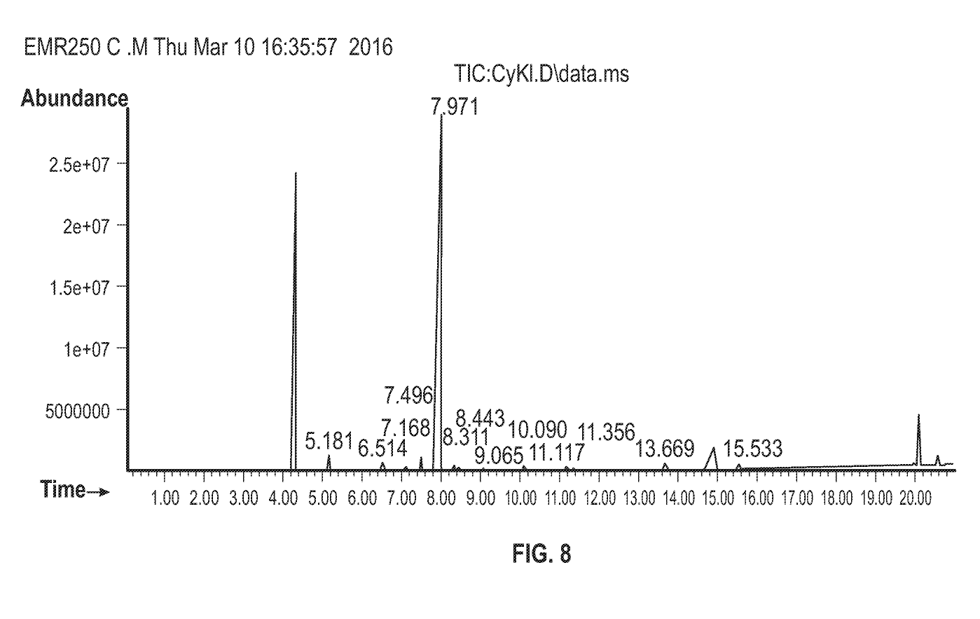

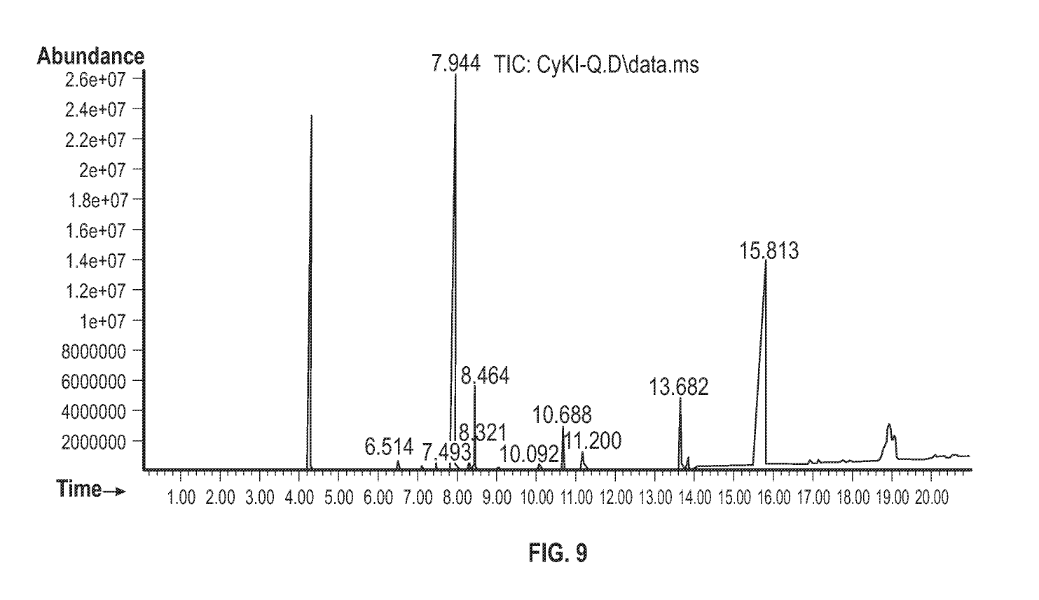

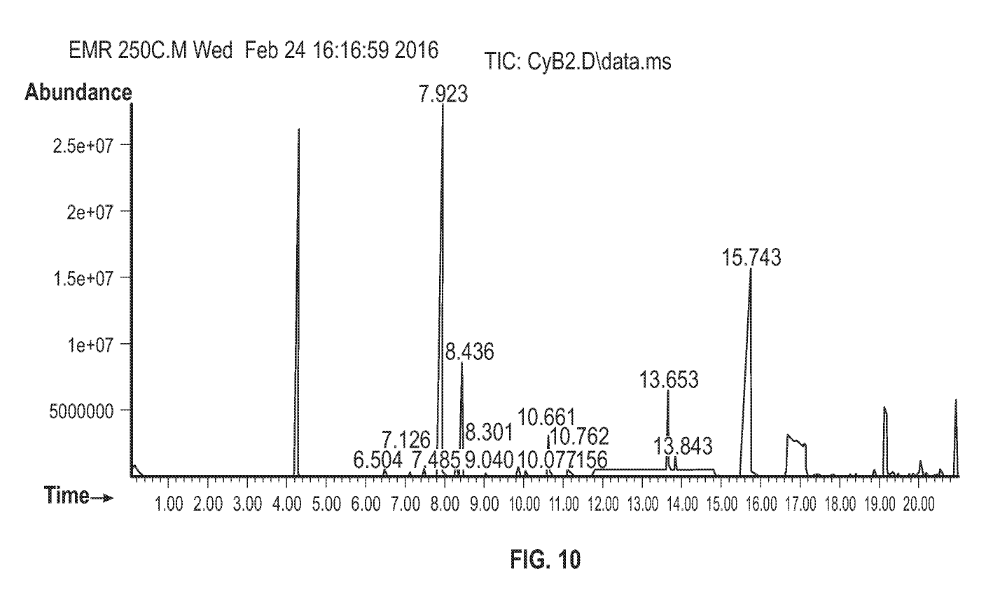

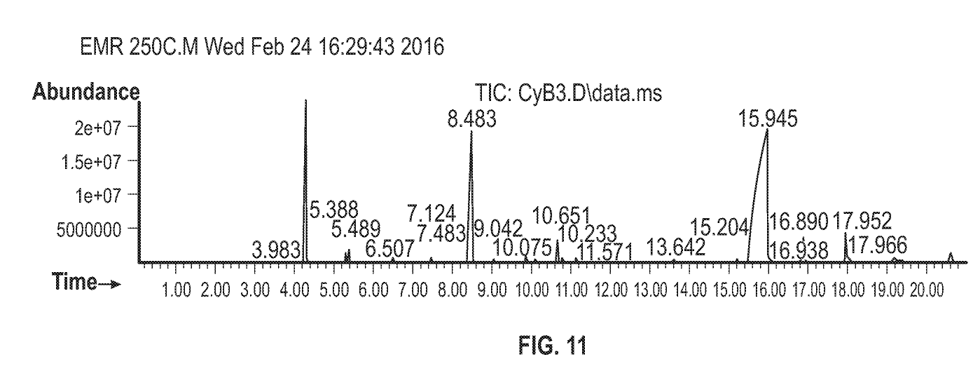

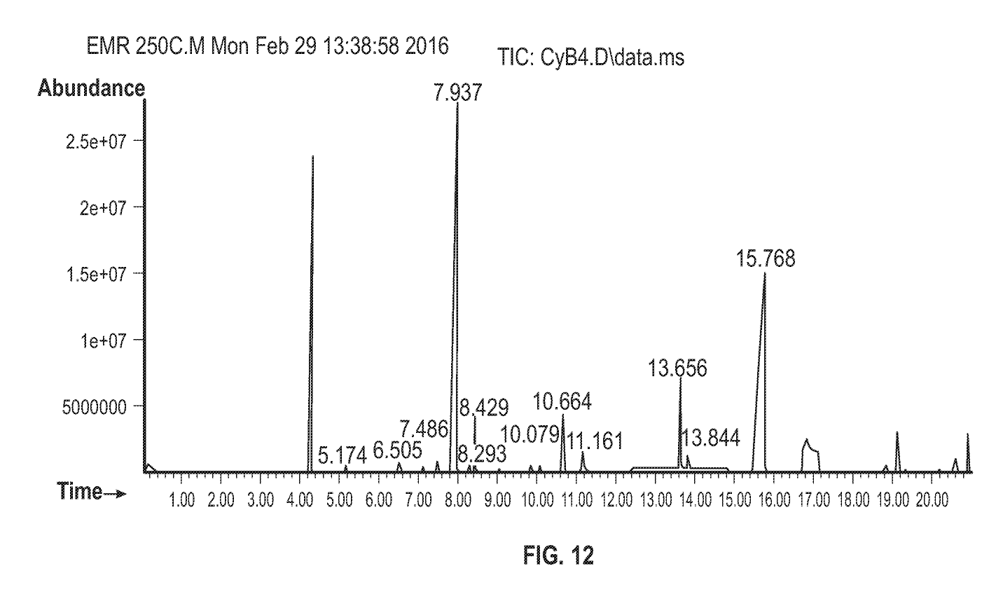

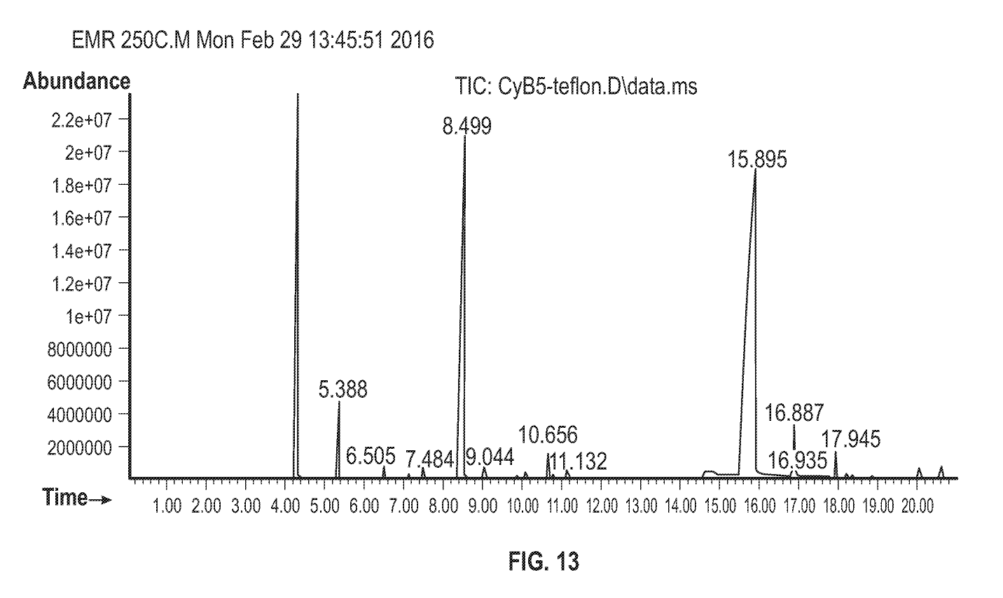

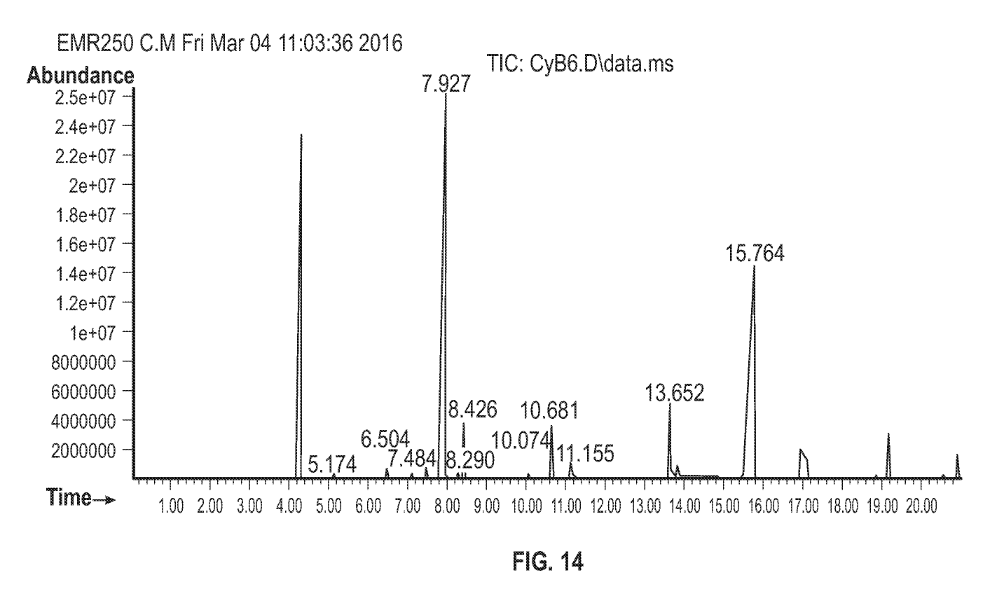

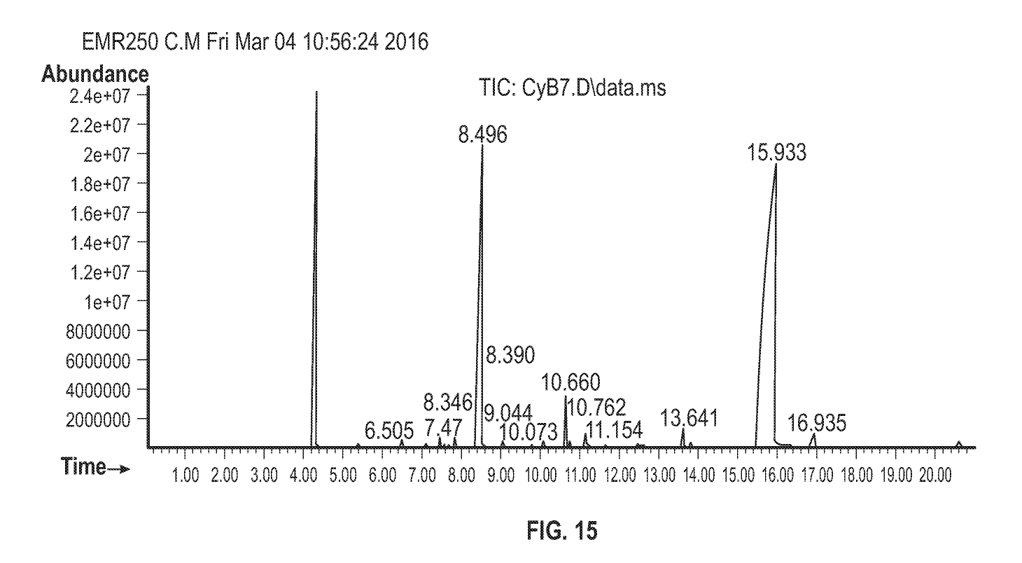

The images presented basically refer to chromatograms obtained from a gas chromatograph coupled with mass spectrometer. As it can be seen from the exampled provided in FIG. 1/19 and for the rest of the presented images, the areas labeled 1-6 refer to: 1) product peak (it normally the widest peak), 2) solvent peak (identified as the thickest peak and similar size as product peak), 3) standard peak (variable size), 4) retention time (refers to the time for each product to be eluted from the column), 5) abundance (level of detection for each product peak, which is proportional to the concentration of each of them), 6) chromatogram data (method and date of emission). It is important to point out that in every chromatogram additional peaks may appear which correspond to byproducts of the reaction, originated likely from the decomposition of main product.

Table 1 shows the number of each chromatogram contained in the final section of the present invention, followed by the reaction of each example presented, using the data from the examples 1 to 3 that are further explained.

TABLE 1

| Figure | Example | Reaction |
|---|---|---|
| 1/19 | Example 1 | DBU 100% |
| 2/19 | | KI/DBU 80:20 |
| 3/19 | | KI/DBU 50:50 |
| 4/19 | | KI/DBU 20:80 |
| 5/19 | | KI/DBU 90:10 |
| 6/19 | | KI/DBU 95:5 |
| 7/19 | | KI 100% |
| 8/19 | Example 3 | KI |
| 9/19 | | KI/chitin |
| 10/19 | | triethyl amine |
| 11/19 | | methyl-imidazole |
| 12/19 | | diethanol-amine |
| 13/19 | | dimetil-aminopyridine |
| 14/19 | | aminoguanidine |
| | | bicarbonate |
| 15/19 | | 1,1,3,3-tetramethyl- |
| | | guanidine |

### DETAILED DESCRIPTION OF THE INVENTION

The present invention shows that is possible to improve the catalytic capacity towards the conversion of CO₂ to cyclic carbonates with a selectivity of 100% and in some cases with quantitative yields, by means of the exploitation of the synergistic effect between an homogeneous and an heterogeneous catalyst supported on a natural polymer, which is capable to increase the catalytic efficiency in the reaction of an alkylene oxide and carbon dioxide. The aforementioned catalyst system does not require the use of organic solvents nor the application of inert operation atmosphere, furthermore it represents minimal risks to the environment because the nitrogen containing catalyst component (DBU) is used in extremely low quantity, and according to the literature, such compound is considered of much lower ecological risk than that of other amines due to its decomposition products.

### Objectives of the Invention

The present invention has as its mean objective the efficient synthesis of cyclic organic carbonates through the reaction of epoxides and carbon dioxide, using catalysts based on alkaline metal halides supported on natural polymers and using organic bases as co-catalysts.

As particular objective there is the use of eco-friendly materials commercially not expensive and with wide availability for their application in chemical transformations of carbon dioxide by a process free of organic solvents.

### EXAMPLES

The reaction experiments were carried out with a reaction zone under variable pressure and constant volume. Such system is made of stainless steel and designed with a couple of three-point flanges and a neoprene packing. The reaction zone is equipped with a magnetic stirrer a manometer, and is placed under a controlled temperature bath.

Preparation of the catalyst system. The preparation of the catalyst is carried out by mixing a mass between 0.1 and 10 g, preferably between 0.5 and 5 g of metal salt, with a polymer mass of 0.1 to 10 g, preferably between 0.5 and 5 g (that is from 20 to 50 wt. %) in water. Such mixture is stirred at a temperature between 10 to 100° C., preferably between 40 and 80° C. at a time between 2 and 24 h, preferably between 10 and 20 h. Afterwards the mixture is evaporated under vacuum at a temperature between 20 to 100° C., preferably from 50 to 80° C. at a time of 1 to 10 h, preferably between 2 and 8 h. An organic nitrogen-containing catalyst I added during the preparation of the reaction mixture at a weight ratio of 1 to 90%, preferably between 20 to 50 wt. percent respective to the metal halide.

Preparation of the cycloaddition reactions. In the reaction zone are added between 5 to 25 g of epoxide and between 0.1 to 5 g of the catalyst system metal salt/natural polymer (between 1 to 15 wt. percent respective to the epoxide). Afterwards 10-200 micrograms organic base are added as co-catalyst (between 20 to 50 wt. percent respective to the metal salt). Such material mixture is subjected to a pressure between 100 and 800 psi, preferably between 200 and 500 psi, so it is further taken to a temperature between 50 and 200° C., preferably between 60 and 150° C. according to the type of epoxide to be tested.

### Example 1

According to the general procedure for the preparation of catalyst, a mass between 0.1 and 10 g, preferably between 0.5 and 5 g of sodium iodide, is mixed with a mass of chitosan between 0.1 and 10 g, preferably between 0.5 and 5 g, in water. Such mixture is stirred at a temperature between 10 to 50° C., preferably between 20 to 40° C. for 0.1 to 10 h, preferably between 0.5 and 5 h. Afterwards, the mixture is evaporated with vacuum at a temperature between 40 and 200° C. preferably between 50 and 100° C. for 2-24 h, preferably between 4 and 12 h. The co-catalyst will be used during the preparation of the reaction.

### Example 2

According to the general procedure for the preparation of the cycloaddition reactions: In the reaction zone between 5 to 25 g of propylene oxide are added and between 0.1 and 5 g of catalyst system sodium iodide/chitin (between 1 to 5 wt. %) After that between 10 and 200 micrograms of triethyl-amine are added as co-catalysts (between 5 and 50 wt. % respective to the metal salt). Such materials are admitted into the reactor and subjected to a pressure between 100 and 800 psi, preferably between 200 and 500 psi so they are further subjected to a temperature between 50 and 200° C., preferably between 60 and 150° C. according to the nature of the epoxide. One the reaction time is accomplished, the reactor is taken to an ice bath and cooled until room temperature, afterwards the gas is released and the mixture is stirred 0.5 h more. The yields are calculated from the reaction mixture using a gas chromatograph coupled with a mass spectrometer.

FIG. 16 shows the influence of the different natural polymer support that can be used in this reaction, in accordance with an embodiment of the present disclosure.

FIG.17 shows the effect of a metal halide over the yield of the reaction of CO₂ and propylene oxide, using chitosan as support, in accordance with an embodiment of the present disclosure.

### Example 2

According to the general procedure for the preparation of the cycloaddition reactions: in the reaction zone, 5 to 25 g of hexylene oxide (1, 2-epoxyhexane) are added and between 0.1 and 5 g of catalyst system potassium iodide/chitin (between 1 and 15 wt. %). Furthermore between 10 and 200 micrograms of DBU as co-catalyst are added (between 5 to 50 wt. percent respective to the metal salt). The mixture of these materials is taken to the reactor and subjected to an initial pressure of 200 to 500 psi and further heated to a temperature between 50 and 200° C., preferably between 60 and 150° C. depending on the nature of the epoxide to be tested. The yields are calculated directly from the reaction mixture using a gas chromatograph coupled with a mass spectrometer.

FIG. 18 shows the influence of the mass composition on the catalyst KI/DBU, in the yield of this reaction, in accordance with an embodiment of the present disclosure.

### Example 3

According to the general procedure for the preparation of the cycloaddition reactions: in the reaction zone between 5 to 25 g of cyclohexene oxide and between 0.1 to 5 g of catalyst system potassium iodide, chitin (between 1 and 15 wt. %) are added. After that, between 10 and 200 micrograms of DBU area added as co-catalyst (between 5 to 50 wt. % respective to the metal salt). Such mixture is taken to the pressure reactor and subjected to a carbon dioxide pressure of 100-800 psi, preferably between 200 and 500 psi and heated to a temperature of 50 to 200° C., preferably between 60 and 150° C. according to the nature of the epoxide to be tested. After de reaction time, the reactor is taken to an ice bath until it gets room temperature, afterwards the excess CO₂ is released and the mixture is stirred for 0.5 h. the yields are calculated directly from the reaction mixture using a gas chromatograph coupled with a mass spectrometer.

Table 2 shows the influence of the temperature and time parameters for this reaction

TABLE 2

| | Temperature | | Yield |
|---|---|---|---|
| Run | (° C.) | Reaction Time (h) | (%) |
| 1 | 100 | 8 | 57.4 |
| 2 | | 24 | 69.5 |
| 3 | | 48 | 80 |
| 4 | 120 | 8 | 69.3 |
| 5 | | 24 | 83.2 |
| 7 | | 48 | 76.5 |
| 8 | 140 | 8 | 71.8 |
| 9 | | 24 | 61.9 |

Besides verifying that the optimal reaction conditions are reached at a 120° C. and 24 h reaction time, the effect of the utilization of different bases is shown in table 3. From this table, it can be demonstrated that the DBU turns out to be the optimal base for this transformation and therefore for the rest of the CO₂ addition reactions to epoxides.

TABLE 3

| Run | Base | Yield (%) |
|---|---|---|
| 1 | KI | 0.5 |
| 2 | KI/Chitin | 45.9 |
| 3 | DBU | 83.2 |
| 4 | triethyl amine | 47.0 |
| 5 | methyl-imidazole | 74.7 |
| 6 | diethanol-amine | 43.7 |
| 7 | dimetil-aminopyridine | 69.6 |
| 8 | aminoguanidine | 45.9 |
| | bicarbonate | |
| 9 | 1,1,3,3-tetramethyl- | 71.9 |
| | guanidine | |

### Example 4

According to the general procedure for the preparation of the cycloaddition reactions: in the reaction zone are added between 5 and 25 g of epichlorohydrin and between 0.1 and 5 g of catalyst system potassium iodide/cellulose (between 1 and 15 weight %). Afterwards 4-dimethyl-aminopyridine is added (between 10 and 200 micrograms) as co-catalyst (between 5 and 50 weight % respective to the metal salt). The aforementioned materials are taken to the Parr reactor and subjected to a pressure between 100 and 800 psi, preferably between 200 and 500 psi so that it can be heated to a temperature between 50 and 200° C., preferably between 60 and 150° C. according to the type of epoxide to be treated. After the reaction time, the reactor is taken to an ice bath and cooled to room temperature, afterwards the excess gas is released and the reaction mixture is stirred for 0.5 h. the yields are calculated directly from the reaction mixture in a gas chromatograph coupled with a mass spectrometer.

### Example 5

Catalyst Recycling

According to the example 1, the recycling of the catalyst system can be carried our according to the following way:

According to the general procedure for the preparation of the cycloaddition reactions: in the reaction zone are added between 5 and 25 g of propylene oxide and between 0.1 and 5 g of potassium iodide/chitin catalyst system (between 5 and 25 weight %). Afterwards triethyl amine is added (between 10 and 200 micrograms) as co-catalyst (between 5 and 50 weight % respective t the metal salt). The aforementioned materials are taken to a Parr reactor and subjected to a pressure between 100 and 800 psi, preferably between 200 and 500 psi so that it can be heated to a temperature between 50 and 200° C., preferably between 60 and 150° C. according to the type of epoxide to be treated. After the reaction time, the reactor is taken to an ice bath and cooled to room temperature, then the excess gas is released and the reaction mixture is stirred for 0.5 h. The yields are determined directly from the reaction mixture in a gas chromatograph coupled to a mass spectrometer.

Finally the catalyst is filtered off from and washed with ethyl ether and n-butanol and taken to a vacuum oven at 80° C. for 2 h under vacuum.

FIG. 19 shows the yield evolution from the reaction after each catalytic cycle, in accordance with an embodiment of the present disclosure.

1. A hybrid catalyst, or catalyst system, for the chemical transformation of carbon dioxide to organic cyclic carbonates through cycloaddition reactions to epoxides, which is composed by an inorganic catalyst, an organic catalyst and a support constituted by a natural polymer.

2. A hybrid catalyst, or catalyst systems, according to claim 1, composed by an inorganic catalyst such as a halide (Cl⁻, Br⁻, I⁻), preferably bromide or iodide of a metal, preferably although not exclusively alkaline (Li, Na, K, Cs).

3. A hybrid catalyst, or catalyst system, according to claim 1, composed by an organic catalyst, such a nitrogen-containing compound, preferably, but not exclusively DBU, triethyl-amine, methyl-Imidazole, diethanol-amine, 4,4-dimethylaminopyridine, Amino guanidine Bicarbonate, 1,1,3,3-tetramethylguanidine, triethanol-amine, 1,4-diazabiciclo[2,2,2]octane.

4. A hybrid catalyst, or catalyst system, according to claim 1, featured because it contains a support which is basically formed by a natural polymer, preferably, but not exclusively chitin, chitosan, carboxymethyl-cellulose, cellulose and starch.

5. A procedure to prepare a hybrid catalyst, or catalyst system to perform cycloaddition reactions with epoxides, according to claim 1 featured because it contains the following stages: 1) preparation of a reaction mixture in aqueous media of the metal halide with the support formed by the natural polymer on weight ratio between 10 and 80%, preferably between 20 and 50% at a temperature between 20 and 100° C., preferably between 40 and 80° C., between 2 and 24 h, preferably between 10 and 20 h and then drying under vacuum at a temperature between 20 and 100° C., preferably between 50 and 80° C. for 1 to 10 h, preferably between 2 and 8 h; 2) addition of the organic catalyst in the reaction mixture at a weight ratio between 1 and 90%, preferably between 10 and 50%; 3) addition of an epoxide in the reaction zone; 4) Injection of CO₂ at a pressure between 100 and 1000 Psi, preferably between 200 and 600 Psi at a temperature between 50 and 200° C., preferably between 60 and 150° C. for a period of time between 0.5 and 8 h, preferably between 1 and 5 h.

6. A process to prepare a hybrid catalyst or catalyst system to perform CO₂ cycloaddition reactions to epoxides, according to claim 5, where the epoxide has the following structure: 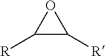
wherein R and R′ could be, although not exclusively, the following groups: hydrogen, phenyl, alkyl, Ar-alkyl, carboxyl, halogen, vinyl, cycloalkyl, cycloalkylene, nitro, epoxy, amine, alkoxide, amide, siliciure, tiol, nitrile, imidazolil, tiofenil, furanil, piridil, heterociclyl.

7. A process to prepare a hybrid catalyst, or catalyst system to perform CO₂-cycloaddition reactions to epoxides, according to claim 5, where the carbonate product has the following structure: 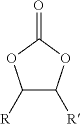
Where R and R′ could be, although not exclusively, the following groups: hydrogen, phenyl, alkyl, Ar-alkyl, carboxyl, halogen, vinyl, cycloalkyl, cycloalkylene, nitro, epoxy, amine, alkoxide, amide, siliciure, tiol, nitrile, imidazolil, tiofenil, furanil, piridil, heterociclyl.
